(19) United States
(12) Patent Application Publication
(10) Pub. No.: US 2020/0016403 A1
(43) Pub. Date: Jan. 16, 2020

(54) IMPLANT DEVICE WITH OPTICAL INTERFACE

(71) Applicant: RETINA IMPLANT AG, Reutlingen (DE)

(72) Inventors: Alfred STETT, Reutlingen (DE); Sandra RUDORF, Reutlingen (DE); Ralf RUDORF, Reutlingen (DE); Henning HELMERS, Freiburg (DE)

(21) Appl. No.: 16/484,018

(22) PCT Filed: Feb. 5, 2018
(86) PCT No.: PCT/EP2018/052748

§ 371 Date: Aug. 6, 2019

(30) Foreign Application Priority Data
Feb. 10, 2017 (DE) 10 2017 102 698.3

(51) Int. Cl. A61N 1/36; A61N 1/05; A61N 1/372; A61N 1/378

(52) U.S. Cl. CPC A61N 1/36046; A61N 1/3787; A61N 1/37217; A61N 1/0543

(57) ABSTRACT

Aspects of the present invention relate to an implant device comprising a first implant part (18) configured for implantation into an eye (10) and a second implant part (21), wherein the eye (10) comprises a sclera (12), and the second implant part (21) is adapted to supply the first implant part (18) with energy transsclerally via an optical interface.

### CROSS-REFERENCE TO RELATED APPLICATIONS

This application is a national stage application under 35 U.S.C. 371 of PCT Application No. PCT/EP2018/052748, filed 5 Feb. 2018, which designated the United States and was published in German as WO 2018/146032 A1, and claims priority to German patent application DE 10 2017 102 698.3, filed 10 Feb. 2017, each of which are incorporated herein by reference in their entirety.

### BACKGROUND OF THE INVENTION

### Field of the Invention

Aspects of the present invention relates to an implant device comprising a first implant part configured for implantation into an eye and a second implant part, wherein the eye comprises a sclera on the inside of which the retina is located, and the second implant part supplies the first implant part with electrical energy via an optical interface, in particular an active retina implant device for electrical stimulation of the retina, wherein the first implant part comprises an array of stimulation electrodes which are configured to provide electrical stimulation signals to cells of the retina, and the first implant part comprises a stimulation chip which is configured to receive image information transmitted optically and generates the electrical stimulation signals.

### Related Prior Art

Such implants can be used for the controlled release of drugs into the eye or as sensor implants for the acquisition of physiological parameters in the eye. However, this disclosure primarily aims at improving retinal implants, especially sub-retinal implants. Such implants are referred to as retinal implants and serve for electrical stimulation of the retina.

An exemplary retina implant is for example known from WO 2005/000395 A1.

The known retinal implant serves to counteract vision loss due to retinal degeneration. The basic idea is to implant a microelectronic stimulation chip into the eye of a patient, which replaces the lost vision by electrical stimulation of nerve cells.

There are two different approaches to how such retinal prostheses can be designed.

The sub-retinal approach described in the aforementioned WO 2005/000395 A1 and for example in EP 0 460 320 A2 uses as part of the first intra-ocular implant part a stimulation chip implanted in the sub-retinal space between the outer retina and the pigment epithelium of the retina, which converts ambient light incident on an array of photodiodes or image elements integrated in the stimulation chip into electrical stimulation signals for nerve cells. These stimulation signals drive an array of stimulation electrodes that stimulate the neurons of the retina with spatially resolved electrical stimulation signals corresponding to the image information “seen” by the array of photodiodes.

This retinal implant thus stimulates the remaining intact neurons of the degenerated retina, i.e. horizontal cells, bipolar cells, amacrine cells and possibly also ganglion cells. The visual image incident on the array of photodiodes or more complex image elements is converted into an electrical stimulation pattern on the stimulation chip. This stimulation pattern then leads to electrical stimulation of neurons, from which the stimulation is then directed to the ganglion cells of the inner retina and from there via the optic nerve into the visual cortex. In other words, the sub-retinal approach exploits the natural interconnection of the former and now degenerated or lost photoreceptors with the ganglion cells in order to provide the visual cortex in the usual way with nerve impulses corresponding to the image seen. The known implant is therefore a replacement for the lost photoreceptors, it converts image information into electrical stimulation patterns.

In contrast, the epi-retinal approach uses a device consisting of an extra-ocular part and an intra-ocular part, which communicate with each other in a suitable way. The extra-ocular part comprises a camera and a microelectronic circuit to encode captured light, i.e. the image information, and transmit it as a stimulation pattern to the intraocular part. The intra-ocular part contains a stimulation chip and an array of stimulation electrodes that contact neurons of the inner retina and thus directly electrically stimulate the ganglion cells located there.

A major problem with known retina implants is the energy supply to the stimulation chip inside the eye.

In epi-retinal implants, the energy for generating the electrical stimulation signals is fed into the eye via cable or inductively. For example, from EP 2 647 358 B1, an epi-retinal implant is known in which the first and second implant parts are connected to each other via cables. On the first implant part, stimulation electrodes and at least one light receiver are arranged as part of the stimulation chip to receive image signals encoding an image captured by an extracorporeal camera.

Even with sub-retinal implants, the energy for generating the electrical stimulation signals cannot be obtained from the incident useful light itself, so that additional external energy is required. This external energy can either be obtained from additional invisible light irradiated into the eye, can be coupled externally for example via a coil, or via a cable into the eye.

Because both sub-retinal and epi-retinal implants must be supplied with external energy, they are also referred to as active retinal implants.

The implant known from the WO 2005/000395 A1 is supplied with electrical energy via irradiated IR light, which is converted into electrical energy on the implant, or wirelessly via inductively coupled HF energy, whereby this externally supplied external energy may contain information for controlling the implant.

EP 2 933 000 A1 describes a retinal implant with an implantable implant part that is inductively via the sclera supplied with signals and data from an external implant part in such that the optical path between the lens of the eye and the retina is not interrupted. Thereby, it can be ensured that patients can make use of their remaining vision.

DE 10 2005 032 989 A1 and US 2002/0198573 A1 each describe a retina implant with an implantable implant part that is inductively supplied with energy via an external coil.

WO 2005/000395 Al in an embodiment uses as a second, extra-ocular implant part extracorporeal IR laser diodes, which illuminate, via the usual optical path, a radiation receiver of the first implant part, which is spatially separated from the stimulation chip in the eye, which has a large number of image elements, each of which supplies electrical stimulation signals to a stimulation electrode.

The implant device known from EP 1 587 578 B1 also uses, as a second implant part, extracorporeal IR laser diodes illuminating a radiation receiver of the first implant part arranged spatially separated from the stimulation chip in the eye, wherein the stimulation chip comprises decoupling means to separate scattered IR light from visible light. This is to avoid problems that can be caused by scattered light falling on the stimulation chip.

However, since wireless retinal implants for applications in humans are not yet available with satisfactory quality, both epi-retinal and sub-retinal implants are currently being used, which are supplied with the required external energy via cables.

WO 2007/121901 A1 describes, for example, a sub-retinal retinal implant in which the external energy and control signals are provided via cable to the stimulation chip implanted in the eye. The cable is attached and fixed to the sclera of the eye to avoid forces on the implant.

Because on the one hand there is usually integrated circuitry on the implants which is operated with DC voltage, and on the other hand there is limited space available on the implant devices themselves, most known implants are directly supplied with a DC voltage. In case of an AC supply voltage, rectifiers required on the implant would take up too much space, in particular due to the required smoothing capacitors, or would not be technically feasible in integrated circuits.

However, the cable-bound transmission of DC voltage in the long term leads to electrolytic decomposition processes in tissue surrounding the cables, so that this type of supply of implants with external energy is also not satisfactory.

WO 2008/037362 A2 therefore suggests to provide the implant with at least one substantially rectangular electrical alternating voltage, which with respect to the tissue mass is on a temporal average almost free from DC voltage. The potential level can be selected such that the supply voltage is at least almost DC voltage-free on average over time. Thereby, the disturbing electrolytic decomposition processes are at least for the most part avoided.

Despite the promising approaches described above for solving the major technological problems associated with epi- and especially sub-retinal retinal implants, the currently available retinal implants may not yet meet all requirements for comprehensive and satisfactory patient care.

### SUMMARY OF THE INVENTION

Against this background, it can be among others an object of the present disclosure to provide an implant device intended for insertion into the eye, in particular an active retinal implant device, which takes these observations into account and avoids or reduces disadvantages of the state of the art, in particular to enable an effective energy coupling with simple construction and with reduced problems regarding scattered light.

According to an aspect of the present disclosure, it is suggested that in the aforementioned implant device, especially in the active retinal implant, the optical interface is configured to transmit the energy transsclerally, i.e., via the sclera.

The inventors of the present application recognized for the first time that the optical characteristics of the sclera are such that transscleral optical transmission of signals and energy is possible.

An advantage here may be that the optical interface is now completely outside the usual optical path, such that little to no stray light problems occur that could interfere with or superimpose the reception of the useful light, i.e. the seen image.

Another advantage may be that the construction of the new implant device is very simple, and the first intra-ocular part of the implant requires little space inside the eye, so that the surgical measures during the implantation of the new implant device do not burden the patient.

Further, because there are no cables running through the sclera, patient comfort and safety may be noticeably increased. The first (intra-ocular) and second (extra-ocular) implant parts are not physically connected to each other, so that furthermore the problems associated with wire-bound energy supply as described above can be effectively avoided without new problems occurring, for example due to stray light.

Moreover, the new implant device may also be used safely within a magnetic resonance technology (MRT) environment.

Many medical implants offer only limited MR safety and MR image compatibility, i.e., they can only be used within defined thresholds in an MRI. Exceeding the threshold values can lead to severe burns, unwanted stimulation of the patient or to a malfunction or functional failure of an active implant (e.g. pacemaker, retina implant).

Since the new implant device uses only a small amount of ferromagnetic or electrically conductive materials, it may provide better MR compatibility than existing implants.

As already mentioned, the new implant device has a simple construction. The second implant part can be supplied with energy via an integrated battery, via cable or inductively and can, for example, be arranged on the side of spectacles such that it can radiate light outside the optical path through the sclera into the eye for the energy supply of the first implant part.

The energy supply of the second implant part can be extracorporeal, e.g. from the glasses or a headband, where a rechargeable battery is provided which supplies the second extra-ocular implant part with energy via cable or inductively.

In an embodiment, the second implant part may be configured to be arranged intracorporeally (within the body).

An advantage may be that also the second implant part may remain permanently on the patient, which on the one hand increases the useful effect for the patient and on the other hand increases the wearing comfort, because thereby the optical interface is fixed and aligned, i.e. above all always ready for operation. In case of a second implant part arranged on a pair of spectacles, non-optimally positioned spectacles could result in the optical interface not being aligned or at least not being optimally aligned and at least not in all situations working perfectly.

The optical interface may also be configured for transmitting data, preferably also for bidirectional data transmission, and the bidirectional optical interface preferably comprises a light transmitter on the first implant part and a light receiver on the second implant part.

Here it may be an advantage that the first implant part can be calibrated by externally transmitted control signals, for which purpose the light beam guided into the eye is modulated in the second implant part and demodulated again in the first implant part.

If the optical interface is configured to be bidirectional, preferably with the transmitter inside the eye and the receiver outside the eye, also data about the function of the first implant part can be read out for the first time in a constructively simple manner via a data light beam. The data can then be processed externally to generate new control signals that are fed into the eye to adapt the first part of the implant to new conditions.

Further, the optical interface may comprise at least one radiation receiver for power supply light arranged on the first implant part and at least one radiation transmitter for the power supply light arranged on the second implant part, and wherein the or each radiation transmitter is configured to be so arranged on the patient such that the or each radiation transmitter can emit the power supply light into the eye such that it can be received by a corresponding radiation receiver at the first implant part, where the power supply light is converted into electrical energy, wherein the power supply light preferably being invisible light.

An advantage here may be that the size and number of transmitters and receivers for the power supply light can be selected such that no critical energy density arises when passing through the sclera. The or each transmitter can be arranged “on the patient”, e.g. in glasses or subcutaneously.

If the energy supply light is non-visible light, for example infrared light in the wavelength range from 780 nm to approx. 3,000 nm, there can be an effective decoupling between the use light, i.e. the image information transmitted on the usual optical path, and the energy supply light.

The second implant part may be configured to be attached externally on the sclera or in the sclera and the radiation receiver of the first implant may be configured to be arranged on the inside of the sclera, preferably in such a way that it is aligned plane-parallel to the radiation transmitter.

An advantage here may be that simple implantation is possible and the energy supply light can be coupled effectively through the eye wall thereby. Furthermore, optimal and permanent alignment between radiation transmitter and radiation receiver is ensured in a simple way.

The radiation transmitter can be placed on the sclera and sutured or otherwise fixed there or inserted into the sclera without protruding into the interior of the eye.

An arrangement or placement of the radiation receiver on the inside of the sclera in the context of the present application may means an arrangement under the choroid (sub-choroidal) or under the retina (sub-retinal).

Further the second implant part may be externally powered via an interface, in particular the interface powering the second implant part is an inductive interface comprising a first, in particular intra-corporeal coil connected to the second implant part and a second, in particular extracorporeal coil, further the second implant part may comprise a circuit device connected between the first coil and the radiation transmitter and providing a supply voltage for the radiation transmitter.

An advantage here may be that no battery may be required in/at the second implant part, which enables longer functional life and problem-free handling. External power can be supplied via a cable, but this does not penetrate the sclera.

If the power supply to the second implant part is inductive, no long cables may be required to connect the intra-corporeal coil to the second implant part, which may provide good wearing comfort. The intracorporeal coil can be implanted subcutaneously, the extracorporeal coil can be placed in glasses or a headband. The circuit device then provides the suitable voltage for the radiation transmitter from the received HF energy.

Thereby the circuit device and the radiation transmitter may be arranged on a common flexible foil (or film) substrate.

The circuit device and the radiation transmitter can be placed on opposite sides of the foil substrate, so that the energy supply light can be radiated unhindered into the sclera and an area is still available for attachment to the sclera. The foil substrate can be at least partially transparent to light in the wavelength range of the energy supply light.

The circuit device and the radiation transmitter may be arranged on the same side of the foil substrate, and preferably an opening is provided in the foil substrate, above which the radiation transmitter is arranged.

An advantage here may be that a foil substrate, which is only provided with circuit elements on one side, can be attached well to the sclera. The foil may be placed on top of the sclera and fixed in a suitable position, whereby the energy supply light passes through the opening or the foil substrate being transparent for the respective wavelength range, into and though the sclera into the interior of the eye and onto the radiation receiver.

The interface powering the second implant part may be configured to transfer power and data from the extracorporeal coil to the second implant part and/or data from the second implant part to the extracorporeal coil.

An advantage here may be that the first and/or second implant part can be calibrated externally, whereby in a bidirectional design of this interface also data on the function of the first and second implant part can be read out.

Furthermore, the first implant part may comprise a flexible foil substrate on which at least the radiation receiver and a stimulation chip may be arranged.

The radiation receiver and stimulation chip can be part of one circuit element or can be manufactured as separate components and then suitably connected to each other. However, a simple implantability and a simpler construction result with the foil substrate. The array of stimulation electrodes may be part of the stimulation chip or separately provided on the foil substrate. A flexible foil substrate may be passed through the retina if the radiation receiver and the stimulation chip are to or must be arranged on different sides of the retina, this applies to sub-retinal and possibly also epi-retinal arrangement of the stimulation chip.

If the radiation receiver and stimulation chip are located on opposite sides of the foil substrate, light will pass unhindered from the sclera to the radiation receiver, but an area of the foil is still available for attachment to the sclera.

The stimulation chip and the radiation receiver may be arranged on the same side of the foil substrate and if an opening is preferably provided in the foil substrate above which the radiation receiver is arranged.

An advantage here may be that the first part of the implant can be easily placed in the eye, the foil substrate is placed on the inside of the sclera and through the opening the light from the sclera passes into the radiation receiver.

On the one hand, the device may comprise an extracorporeal camera which converts incident ambient light into the image information that is transmitted optically to the stimulation chip and, on the other hand, the stimulation chip may comprise a plurality of image elements which are adapted to receive ambient light incident on the eye as image information.

An advantage here may be that the new type of energy supply can be used for both an epi-retinal and a sub-retinal device.

The stimulation chip may be connected to at least one counter electrode via which the stimulation signals from the stimulation electrodes flow back into the stimulation chip.

An advantage here may be that the current path is effectively closed. The counter electrode can be a stimulation electrode of the array that is not used for stimulation, an electrode to be positioned separately in/on the eye, which is connected to the array or the stimulation chip via a cable, and/or arranged on the substrate foil, on the stimulation chip and/or on the radiation receiver.

Further advantages will be apparent from the description and the attached drawings.

It is to be understood that the features mentioned above and the features to be explained below can be used not only in the indicated combination, but also in other combinations or in alone, without leaving the scope of the present invention.

### BRIEF DESCRIPTION OF THE DRAWINGS

Exemplary embodiments of aspects of the invention are illustrated in the drawings and are explained in more detail in the following description.

FIG. 1 shows a schematic representation of the arrangement of the new retinal implant device in an eye, not drawing to scale;

FIG. 2 shows a schematic representation of the interaction of the first and second implant parts of the retinal implant device of FIG. 1, not drawn to scale;

FIG. 3 shows a schematic representation of a top view of the first implant part from FIG. 2, also not drawn to scale;

FIG. 4 shows a schematic representation of a further arrangement of the second implant part, not drawn to scale;

FIG. 5 shows a schematic representation of a third arrangement of the second implant part, not drawn to scale;

FIG. 6 shows a schematic representation of a further arrangement of the first implant part, not drawn to scale;

FIG. 7 shows a schematic representation of a third arrangement of the first implant part, not drawn to scale; and

FIG. 8 shows a schematic representation of a fourth arrangement of the first implant part, not drawn to scale.

### DESCRIPTION OF PREFERRED EMBODIMENTS

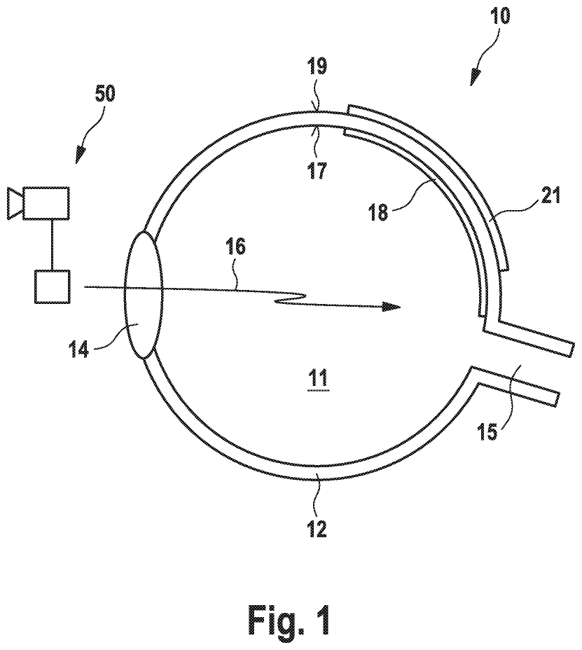

FIG. 1 shows an eye 10 with a vitreous body 11 surrounded by a sclera 12 in which a lens 14 can be seen. From sclera 12, an optic nerve 15 branches off approximately opposite lens 14, via which eye 10 is connected to the visual cortex in the brain.

Through the lens 14 via the usual optical path 16 light enters the interior of the eye, where the sclera 12 carries a first implant part 18 on its inner side 17.

The first implant part 18 interacts in a manner yet to be described with a second implant part 21 arranged on an outer side 19 of the sclera 12.

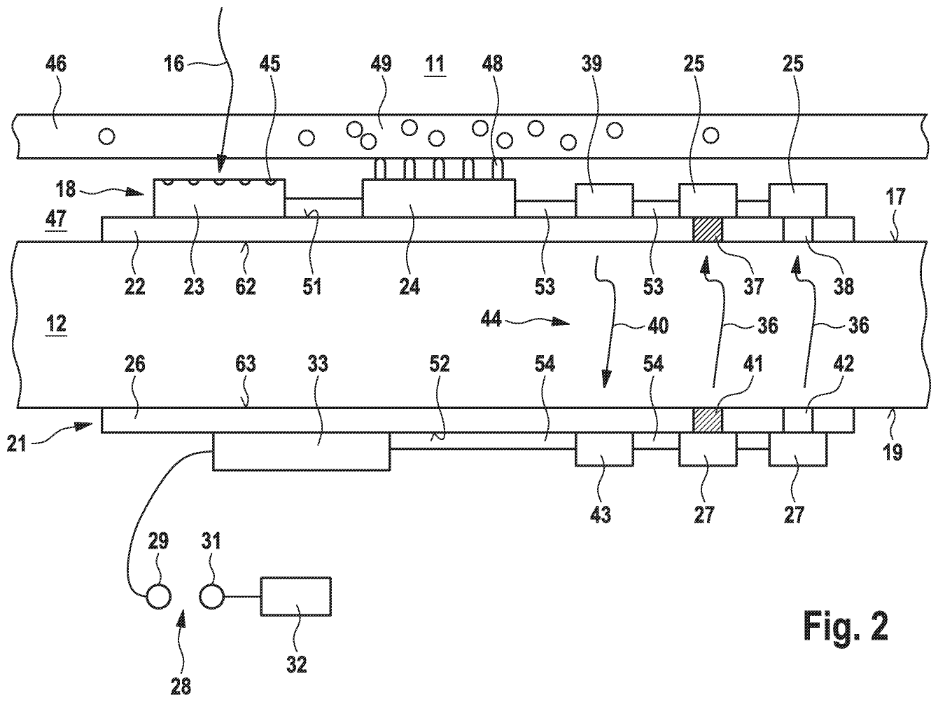

In FIG. 2, the first implant part 18 and the second implant part 21 are arranged on the opposite sides 17 and 19 of the sclera 12 and aligned plane-parallel to each other.

It should be noted that both the diagram of FIG. 1 and the diagram of FIG. 2 are only exemplary and schematic, they do not correspond exactly to the biological or technical conditions.

The first implant part 18 has a flexible foil substrate 22 which, in the example of FIG. 2, is arranged on the inside 17 of the sclera 12.

A stimulation chip 23 is placed on the foil substrate 22 facing away from the inner side 17, i.e. facing the glass body 11, which receives and processes, in a manner yet to be described, image information that reaches the eye 10 via the optical path 16.

This stimulation chip 23 generates electrical stimulation signals which are provided in the eye via an array 24 of stimulation electrodes. In addition to the stimulation chip 23 and the array 24 of stimulation electrodes, there are two radiation receivers 25, which serve as the energy supply of the first implant part 18 in a way still to be described.

In FIG. 2 the second implant part 21 is shown on the outside 19 of the sclera 12, which has a flexible foil substrate 26 on which two radiation transmitters 27 are arranged.

In FIG. 2 the two implant parts 18, 21 are, similar to the sclera 12, shown as plane elements for simplification. In reality, the sclera is curved and the implant parts 18, 21 adapt to the curved shape of the sclera 12 due to the flexibility of the foil substrates 22, 26, but are nevertheless aligned plane-parallel to each other.

The power supply of the second implant part 21 is provided via an inductive interface 28, which has a first, intracorporeal coil 29 as well as a second, extracorporeal coil 31, which is connected to a control unit 32.

The intra-corporeal coil 29 can for example be arranged subcutaneously, while the extra-corporeal coil together with the control unit 32 is for example arranged on spectacles worn by the patient or a headband. A rechargeable battery can be inserted in the control unit 32.

On the flexible foil substrate 26 of the second implant part 21 there is a circuit device 33 connected to the intracorporeal coil 29, which provides a supply voltage for the radiation transmitters 27.

The two radiation transmitters 27 emit energy supply light 36 preferably in the infrared wavelength range from 780 nm to 3,000 nm. This energy supply light 36 is received by the radiation receivers 25 of the first implant part 18 and converted there into a supply voltage for the first implant part 18.

The wavelength of the energy supply light 36 is selected such that this light can easily penetrate the sklera 12. It is known that the sklera 12 is transmissive to light, especially in the infrared range.

In order for the power supply light 36 to reach the radiation receivers 25, the flexible foil substrate 22 has either a transparent area 37 or an opening 38, each of which is located below the radiation receiver. The flexible foil substrate 22 itself can also be completely or partially transparent.

On the flexible foil substrate 22 of the first implant part, a light transmitter 39 is shown, which interacts via a data light beam 40 with a light receiver 43 on the flexible foil substrate 26 of the second implant part 21.

Also in the flexible foil substrate 26, either a transparent area 41 or an opening 42 is provided below the radiation transmitters 27, such that the energy supply light 36 can pass through the flexible foil substrate 26 into the sclera 12 and from there through the flexible foil substrate 22 to the radiation receivers 25. The flexible foil substrate 26 itself can also be completely or partially transparent.

The data light beam 40 penetrates the foil substrates 22, 26, which are transparent at least below the light transmitter 39 and light receiver 43 for light at the wavelength of the data light beam 40, which also lies within the range from 780 nm to 3,000 nm.

Thereby an optical interface 44 is formed, via which on the one hand power supply light 36 from the radiation transmitters 27 reaches the radiation receivers 25, whereby the second implant part 21 transsclerally supplies the first implant part 18 with energy, and on the other hand by modulating the light beam of the power supply light 36 signals and information can be transmitted to the first implant part 18.

The optical interface 44 is also bidirectional, the light transmitter 39 and the light receiver 43 enable data and information to be transmitted optically from the first implant part 18 to the second implant part 21 via the correspondingly modulated data light beam 40.

In the same way, the inductive interface 28 is adapted bidirectionally such that it not only transmits energy from the control unit 32 to the second implant part 21, but can also transmit and process information received from the second implant part 21 via the data light beam 40 from the first implant part 18.

In addition to energy, information and control signals can also be transmitted to the second implant part 21 via the inductive interface 28, which are then transmitted to the first implant part 18 by modulation onto the light beam of the energy supply light.

Thereby it is possible to exchange data and information between the first implant part 18 and the second implant part 21 via the data light beam 40 and the light beam of the power supply light 36, such that the operation of the first implant part 18 can be adapted to the respective physiological or other conditions which are measured inside the eye 10 and which reach the control unit 32 via the optical interface 44 and the inductive interface 28, where this information is then converted into control signals which reach the first implant part 18 in the opposite direction and adjust the same accordingly.

According to FIG. 2, a plurality of image elements 45 are arranged in the stimulation chip 23, which receive image information reaching the eye 10 via the usual optical path 16.

As already mentioned at the beginning, this image information can either correspond to the naturally seen image, it is then a sub-retinal implant as shown in FIG. 2.

In this case the picture elements 45 each comprise a photodiode, which converts the locally incident light into a current, which is then converted into an electrical stimulation pulse in an amplifier and possibly downstream electronics, as described in detail in WO 2005/000395 A1 and the documents cited in this publication. Image elements 45 then receive the incident use light as spatially resolved image information.

In the illustration of FIG. 2, the first implant part 18 is arranged in the subretinal slit 47 formed between the sclera 12 and the retina 46. The position of the choroid is not shown in FIG. 2 for reasons of clarity.

The optical, i.e. spatially resolved image that reaches the stimulation chip 23 via the optical path 16 is converted into stimulation signals that are transmitted to cells 49 of the retina 46 via stimulation electrodes 48, which are arranged in the array 24.

Thereby the active retinal implant device formed by the first implant part 18 and the second implant part 21 serves to at least partially restore a patient's vision loss, as described above.

As an alternative to a sub-retinal implant, the retinal implant device can also be used as an epi-retinal implant, in which case then, via the usual optical path 16, image information from a camera sketched in FIG. 1 at 50 is transmitted. The camera 50 essentially takes over the function of the image elements 45 by converting the image incident from the outside onto the camera into electrical image information, which is then transmitted via the optical path 16 to the stimulation chip 23, which then converts this information back into spatially resolved electrical stimulation pulses.

In FIG. 2 it can further be seen that the stimulation chip 23 as well as the array 24 of stimulation electrodes 48 together with the radiation receivers 25 and the light transmitter 39 are arranged on an upper side 51 of the flexible foil substrate 22.

Likewise, the circuit device 33, the radiation transmitter 27 and the light receiver 43 are arranged on an upper side 52 of the flexible foil substrate 26.

On the upper side 51, conductive traces 53 are shown, which connect the stimulation chip 23 with the array 24 of stimulation electrodes 48 and the radiation receivers 25 as well as the light transmitter 39 in a known manner.

Also on the upper side there are 52 conductive traces 54, which connect the circuit device 33 with the radiation transmitters 27 as well as the light receiver 43.

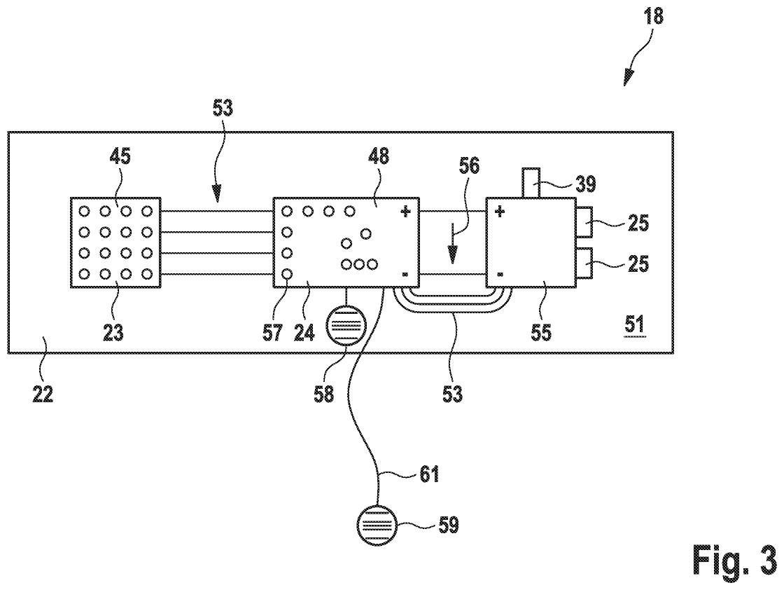

FIG. 3 shows a schematic top view of the first implant part 18, wherein further or next to the stimulation chip 23 and the array of stimulation electrodes 48 on the upper side 51 a control unit 55 is arranged, which is suitably connected to the radiation receivers 25 and the light transmitter 39 and generates a supply voltage 56 for the first implant part 18 from the received power supply light 36.

The stimulation chip 23, array 24 and control unit 55 are connected to each other via conductive traces 53. The components 23, 24, 53, 55, 25, 39 can be arranged on the flexible foil substrate 22 or laminated between two such films or foils.

Further, the control unit 55 extracts information and control signals from the power supply light 36 and, on the other hand, encodes data and information available in the first implant part 18 in the data beam 40 transmitted by the light transmitter 39.

It should further be mentioned that the implant device shown in FIGS. 2 and 3 comprises two radiation transmitters 27 and two radiation receivers 25, although this is only an example. Depending on the power consumption of the respective implant device, the optical interface 44 can also have only one radiation transmitter 27 and only one radiation receiver 25, or more than two radiation transmitters 27 and radiation receivers 25. With the number of these transmitter/receiver pairs the energy density in the sclera 12 can be kept so low that sufficient energy can be transmitted to the first implant part 18 via the optical interface 44, while on the other hand avoiding local overheating of the sclera 12.

It should further be noted that the first implant part 18 does not necessarily have to be a retinal implant device; instead of the stimulation chip 23 and the array 24, the first implant part 18 may also comprise dosing devices for drugs and/or sensors that detect physiological states in the eye.

The dosing device is controlled by control signals which are transmitted via the light beam of the power supply light 36, whereby the data of the sensors are transmitted via the data light beam 40 from the eye 10.

In any case, it is important that the first implant part 18 and the second implant part 21 can exchange data and control signals bidirectionally via the optical interface 44, wherein the first implant part 18 is powered transsclerally by the second implant part 21.

If the first part of the implant is a retina implant, at least one counter electrode must be provided for the stimulation signals emitted by the stimulation electrodes to close the electric circuit.

FIG. 3 shows three examples of the arrangement of such counter electrodes. On the one hand, a counter electrode 57 can be part of the array 24 of stimulation electrodes 48, which means nothing more than that some of the electrodes in the array 24 are connected as counter electrodes 57.

It is also possible to provide a large-area counter electrode 58 on the upper side 51 or on the lower side of the flexible foil substrate 22.

Alternatively and/or additionally, it can also be useful to connect a counter electrode 59 to the array 24 via a flexible cable 61, whereby this flexible cable 61 can then be so long that the counter electrode 59 can be arranged on the outer side 19 of the sclera 12.

The counter electrode is connected to the stimulation chip 23 or the control unit 55 via the array 24 so that the circuit is closed. Depending on the arrangement of the counter-electrode, different current paths result for the stimulation signals inside the eye.

In addition/alternatively, it is also possible that the stimulation chip 23, the array 24 and possibly also the control unit 55 are implemented as integrated circuit chips which can be arranged one on top of the other or arranged next to each other. Counter electrodes can also be provided on the outside of these circuit chips.

In the arrangement according to FIG. 2, the flexible foil substrate 22 lies with its lower side 62 flat on the inside 17 of the sclera, while the flexible foil substrate 26 lies flat with its underside 63 flat on the outside 19 of the sclera 12. This allows good positioning and fixation of the first implant part 18 and the second implant part 21 on the opposite sides 17, 19 of the sclera 12, so that a plane-parallel arrangement of the first implant part 18 and the second implant part 21 is achieved.

This plane-parallel arrangement of the two implant parts 18, 21 in relation to each other enables precise alignment of the transmitters and receivers of the optical interface 44 in relation to each other, so that a very effective and low-loss transmission of energy and data is possible.

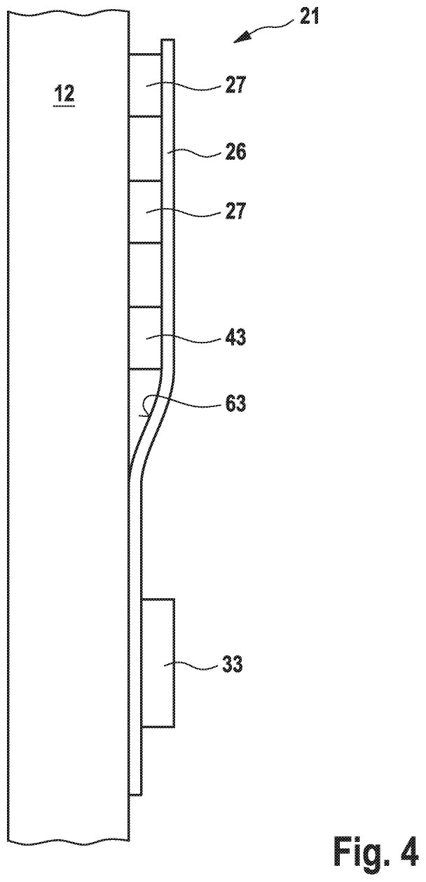

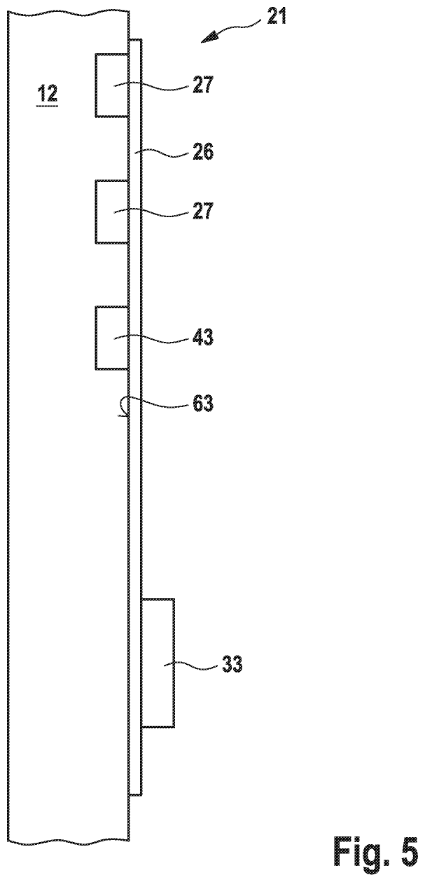

However, it is also possible to place the radiation transmitters 27 and the light receiver 43 on the lower side 63 of the flexible foil substrate 26, as shown in FIG. 4. This arrangement then also makes it possible to insert the radiation transmitters 27 and possibly the light receiver 43 at least partially into the sclera, as can be seen in FIG. 5.

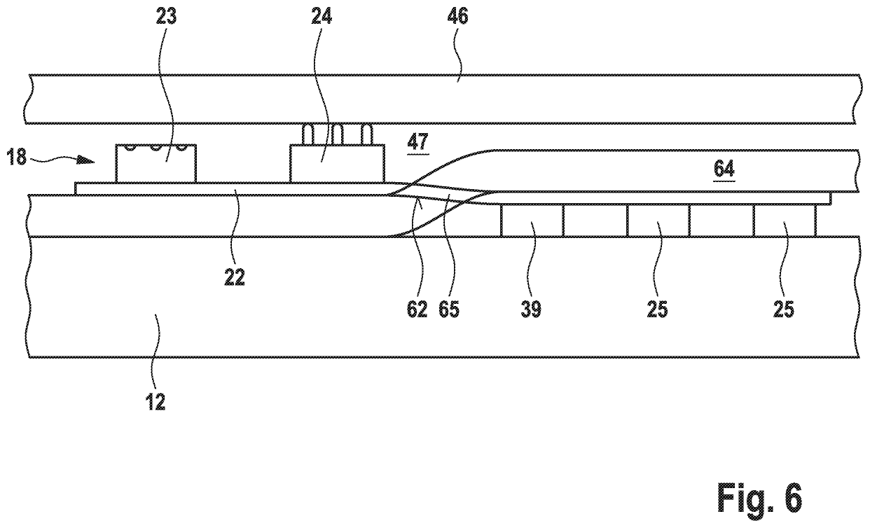

It is also possible in the first implant part 18 to arrange the radiation receiver 25 and optionally the light transmitter 39 on the lower side 62 of the flexible foil substrate 22, as shown in FIG. 6, where the radiation receiver 25 is arranged under the choroid 64, i.e., sub-choroidally. The radiation receiver is then connected to the remaining first implant part 18 via a trans-choroidal conductor band 65.

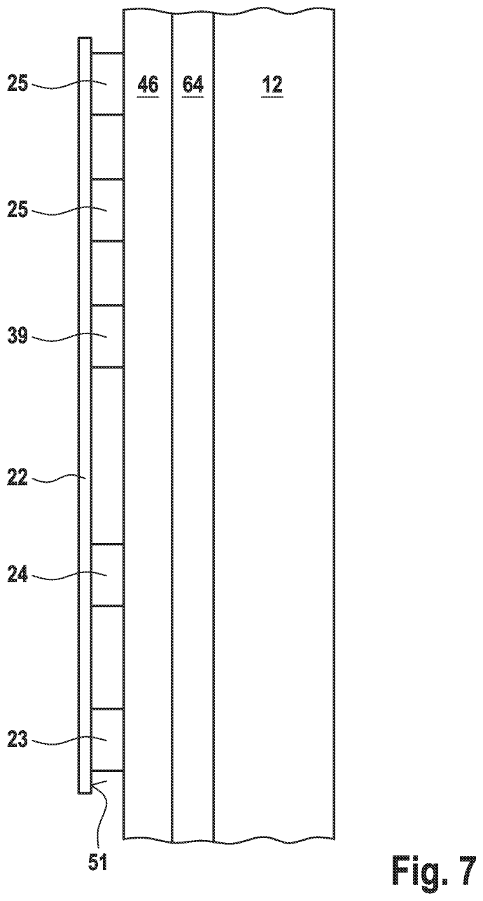

FIG. 7 shows an further arrangement in which the stimulation chip 23 and the array 24 of stimulation electrodes are arranged epi-retinally. In the example of FIG. 7, the radiation receivers 25 and the control unit 55 are again located on the upper side 51 of the flexible foil substrate 22, such that the first implant part 18 is placed on the retina 46.

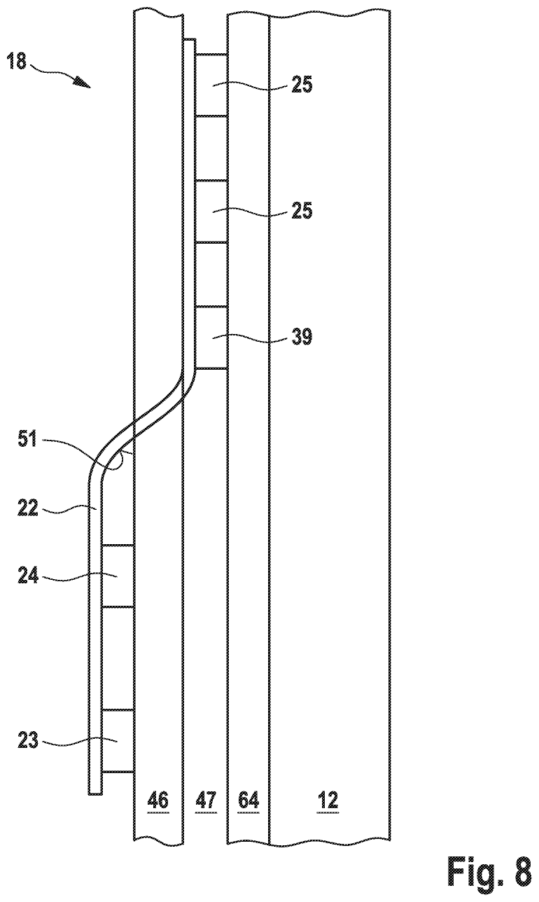

In the epi-retinal arrangement, the radiation receivers 25 and the light transmitter 39 can also be arranged in the sub-retinal slit 47. The flexible foil substrate 22 must then penetrate retina 46 as shown in FIG. 8.

1. An implant device comprising a first implant part configured for implantation into an eye and a second implant part, wherein the eye comprises a sclera on the inside of which the retina is located, and the second implant part is configured to supply the first implant part with electrical energy via an optical interface, wherein the optical interface is configured to transfer the energy transsclerally.

2. The device of claim 1, wherein the device is an active retinal implant device for electrical stimulation of the retina, wherein the first implant part comprises an array of stimulation electrodes which are configured to emit electrical stimulation signals to cells of the retinae, and wherein the first implant part comprises a stimulation chip which is configured to receive image information transmitted optically and which generates the electrical stimulation signals.

3. The device of claim 1, wherein the second implant part is configured to be arranged intracorporeally.

4. The device of claim 1, wherein the optical interface is also configured for data transmission.

5. The device of claim 4, wherein the optical interface is also configured for bidirectional transmission of data.

6. The device of claim 5, wherein the bidirectional optical interface comprises a light transmitter on the first implant part and a light receiver on the second implant part.

7. The device of claim 1, wherein the optical interface comprises at least one radiation receiver for power supply light arranged on the first implant part, and at least one radiation transmitter for the power supply light arranged on the second implant part, and wherein the or each radiation transmitter is configured to be arranged on the patient such that the or each radiation transmitter can emit the power supply light into the eye such that it can be received by a corresponding radiation receiver on the first implant part, where the power supply light is converted into electrical energy, wherein the power supply light is preferably invisible light.

8. The device of claim 7, wherein the second implant part is configured to be attached externally on the sclera or in the sclera, and wherein the radiation receiver of the first implant is configured to be arranged on the inside of the sclera, preferably such that it is aligned plane-parallel to the radiation transmitter.

9. The device of claim 1, wherein the second implant part is externally supplied with energy via an interface.

10. The device of claims 9, wherein the interface supplying energy to the second implant part is an inductive interface comprising a first, preferably intracorporeal coil connected to the second implant part, and a second, preferably extracorporeal coil.

11. The device of claim 10, wherein the second implant part comprises a circuit device which is connected between the first coil and the radiation transmitter and provides a supply voltage for the radiation transmitter.

12. The device of claim 11, wherein the circuit device and the radiation transmitter are arranged on a common flexible foil substrate.

13. The device of claim 12, wherein the circuit device and the radiation transmitter are arranged on the same side of the foil substrate, and in the foil substrate preferably an opening is provided above which the radiation transmitter is arranged.

14. The device of claim 10, wherein the interface powering the second implant part is configured to transmit power and data from the extracorporeal coil to the second implant part.

15. The device of claim 14, wherein the interface powering the second implant part is adapted to transmit data from the second implant part to the extracorporeal coil.

16. The device of claim 7, wherein the first implant part comprises a flexible foil substrate on which at least the radiation receiver and a stimulation chip are arranged.

17. The device of claim 16, wherein the stimulation chip and the radiation receiver are arranged on the same side of the foil substrate, and in the foil substrate preferably an opening is provided, above which the radiation receiver is arranged.

18. The device of claim 2, wherein the device comprises an extracorporeal camera which converts incident ambient light into the image information that is transmitted optically to the stimulation chip.

19. The device of claim 2, wherein the stimulation chip comprises a plurality of picture elements which are configured to receive ambient light incident into the eye as image information.

20. The device of claim 2, wherein the stimulation chip is connected to at least one counter electrode via which the stimulation signals flow back from the stimulation electrodes into the stimulation chip.
